(19) United States
(12) Patent Application Publication
(10) Pub. No.: US 2025/0235573 A1
(43) Pub. Date: Jul. 24, 2025

(54) Method for Fragrancing an Interior of a Vehicle

(71) Applicant: Bayerische Motoren Werke Aktiengesellschaft, Muenchen (DE)

(72) Inventor: Annabelle COFFINET, Muenchen (DE)

(21) Appl. No.: 18/832,635

(22) PCT Filed: Mar. 24, 2023
(86) PCT No.: PCT/EP2023/057713

§ 371 Date: Jul. 24, 2024

(30) Foreign Application Priority Data
Apr. 12, 2022 (DE) 10 2022 108 957.6

(51) Int. Cl. A61L 9/04; B60H 3/00

(52) U.S. Cl. CPC A61L 9/04; B60H 3/0035; A61L 2209/111

(57) ABSTRACT

A method for fragrancing an interior of a vehicle based on fragrances captured from the surroundings of the vehicle is provided. In case A), a fragrance entering the interior is manually captured by an occupant of the vehicle by way of a fragrance capture apparatus, in which the captured fragrance is stored and/or analyzed by an external device or directly in the fragrance capture apparatus and is categorized as desirable or undesirable. Alternatively or additionally, in case B), a fragrance outside the vehicle is automatically captured and analyzed by way of a fragrance capture apparatus, wherein the captured fragrance is categorized as desirable or undesirable and, based on the categorization, a fragrance categorized as desirable is allowed to enter the interior of the vehicle or a fragrance categorized as undesirable is prevented from entering the vehicle.

### BACKGROUND AND SUMMARY

The invention relates to a method for fragrancing an interior of a vehicle.

There are already fragrance diffusers in vehicles with the prime objective of diffusing a pleasant smell in the vehicle interior. There are also already known systems that prevent air from being supplied to the interior of the vehicle if harmful gases are measured outside the vehicle, as may happen for example when there is a tailback in a tunnel. Otherwise, so far there are not any known systems that work broadly in the vehicle with the detection of smells.

It is therefore an object of this invention to provide an improved method for fragrancing an interior of a vehicle. This object is achieved according to the claimed invention.

A method is provided for fragrancing an interior of a vehicle on the basis of smells captured from the surroundings of the vehicle, where case A) manual capturing of a smell entering the interior is performed by an occupant of the vehicle by way of a smell capturing device, in which the captured smell is stored and/or analyzed and categorized as desired or undesired by an external facility or directly in the smell capturing device. Alternatively or additionally, in case B) automatic capturing and analysis of a smell occurring outside the vehicle are performed by way of a smell capturing device, with the captured smell being categorized as desired or undesired and, on the basis of the categorization, a smell categorized as desired being allowed to enter the interior of the vehicle or a smell categorized as undesired being prevented from entering.

In one configuration it is provided that in case A) automatic capturing of a smell outside the vehicle may be additionally performed, the smell is compared with specified smells and, on the basis of this comparison, the smell is allowed to enter or prevented from entering the interior of the vehicle.

In one configuration it is provided that a specimen of the stored smell is created for duplication. The specimen is advantageously integrated in a fragrance diffuser for use in the vehicle.

In one configuration it is provided that in case B) the captured and analyzed smell is compared with specified smells and, on the basis of this comparison, the smell is allowed to enter or prevented from entering the interior of the vehicle.

In one configuration it is provided that the air supply into the interior of the vehicle is opened or increased if a smell categorized as desired has been captured.

In one configuration it is provided that storing of the position of the captured smell is performed.

In one configuration it is provided that a route which includes as many sections as possible having smells categorized as desired is calculated for a specified destination.

In one configuration it is provided that positions of smells categorized as desired or as undesired are provided as information outside the vehicle.

Also proposed is a vehicle, having a smell capturing device which is designed to store and/or analyze smells from outside the vehicle, and a processing facility for carrying out the method for fragrancing an interior of a vehicle on the basis of smells captured from the surroundings of the vehicle.

Further features and advantages of the invention will emerge from the description of exemplary embodiments of the invention that follows, on the basis of the drawings, which show details according to the invention, and from the claims. The individual features may be implemented in a variant of the invention each individually on their own or together in any desired combination.

Preferred embodiments of the invention are explained in more detail below on the basis of the appended drawings.

### BRIEF DESCRIPTION OF THE DRAWINGS

FIG. 1 shows a schematic representation of important components for carrying out the method according to one embodiment of the present invention.

FIG. 2 shows a schematic representation of important components for carrying out the method according to a further embodiment of the present invention.

FIG. 3 shows a schematic representation of important components for carrying out the method according to a further embodiment of the present invention.

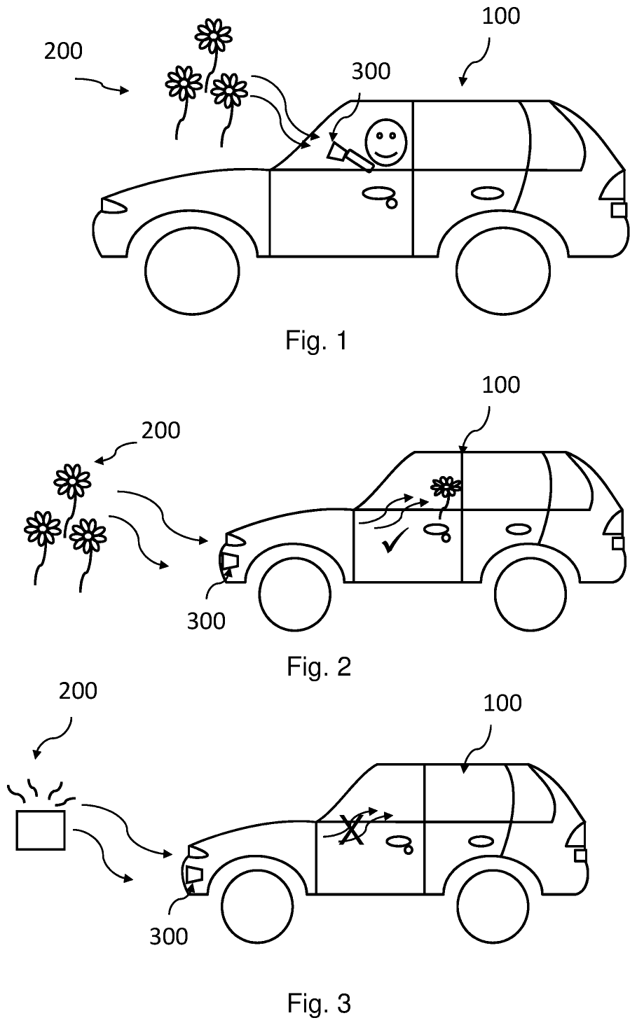

### DETAILED DESCRIPTION OF THE DRAWINGS

In the following descriptions of the figures, the same elements or functions are provided with the same designations.

The aim of the invention is to capture smells 200 occurring outside and/or inside the vehicle 100 and, depending on the captured smell 200, perform an action. The term smells 200 should be understood here as meaning not only fragrant smells 200 but also smells 200 that people find unpleasant, known as odors.

The proposed method for fragrancing an interior of a vehicle 100 serves on the one hand for making the experience of a smell repeatable. On the other hand, the same technology can be used to exclude undesired smells 200 from the interior of the vehicle 100.

To achieve this, a smell capturing device 300 is provided in the interior and/or on the outer region of the vehicle 100. The smell capturing device 300 may be formed here as a device which has a bell for receiving and storing a smell 200. The bell is in this case held in the direction of the smell 200 and the smell is sucked into the bell, for example by way of a pump, and bound by an appropriate material, for example a resin. This material with the smell 200 bound to it (or the molecules of the smell) can then be removed and for example passed on to a laboratory for analysis. From the analysis, a specimen of the smell can then be produced, duplicated and used as a fragrance diffuser in the vehicle 100. Smell capturing devices 300 that use such a bell are already known and are therefore not described any further.

This embodiment requires manual operation of the smell capturing device 300. In this case, the smell capturing device 300 is advantageously provided in the interior of the vehicle 100 and within reach of an occupant, for example the front passenger. For example, it may be provided in a cup holder in the region of the center console or the dashboard. As soon as the occupant perceives a smell 100 that they would for example like to smell again later, or no longer smell in the vehicle 100, the occupant can capture the smell by way of the smell capturing device 300, store it and have it analyzed externally.

If the stored smell 200 is a smell 200 that the occupant would like to experience again, that is to say is a desired smell 200, this smell 200 can be duplicated on the basis of the analysis and used in a fragrance diffuser both in the vehicle 100 and for example at home.

In addition to the manual capturing of a smell 200, automatic capturing of smells outside the vehicle may also be performed, for example by a corresponding vehicle sensor such as an electronic nose as a smell capturing device 300.

Consequently, both desired smells 200 and undesired smells 200, also referred to as odors, may be stored for example in the vehicle 100 as correspondingly desired or undesired. As soon as the vehicle sensor (smell capturing device 300) captures a smell 200 categorized as desired or undesired, an action may be stored in the vehicle 100 as a reaction to the smell. For example, closing of the ventilation may be performed from the outside, that is to say that no air from outside the vehicle 100 is let in if a smell categorized as undesired is captured (indicated in FIG. 3). In the case where a smell 200 categorized as desired is captured, the ventilation may be opened or the air flow increased, in order to let the smell 200 into the interior more quickly and/or more intensively (indicated in FIG. 2).

The position of the vehicle 100 at which the smell 200 was captured by the smell capturing device 300 may also be stored, preferably in a navigation system of the vehicle 100. This may take place automatically whenever the smell capturing device 300 (for example smell capturing bell) is used, or be triggered by the occupant using the smell capturing bell.

A smell 200 entering the interior that can be recorded by way of the smell capturing device 300 (for example smell capturing bell) may be a smell 200 entering from the surroundings of the vehicle 100 by way of the air-conditioning system or an open window.

The smell capturing device 300 may also be formed exclusively such that automatic capturing of a smell 200 can be performed. Such a smell capturing device 300 (for example vehicle sensor, chip) is advantageously provided outside the vehicle 100 and is used for the early detection of a smell 200. This is advisable in particular if a smell 200 categorized as undesired is detected, for example an odor of liquid manure.

If it is formed as a corresponding sensor, the smell capturing device 300 may also be capable of performing an analysis of a captured smell 200. In this case, a captured smell 200 may also be categorized as undesired or desired, for example by directing an inquiry to an occupant. Smells 200 categorized as undesired or desired may also be stored in the vehicle 100, so that a permanent comparison of currently captured smells 200 can take place and an action be performed on the basis of this comparison. The action may be the same as described above, that is to say closing of the ventilation or opening and/or increasing of the ventilation.

Smell capturing devices 300 that can perform a direct evaluation of a captured smell 200 are likewise already known and are also referred to as an electronic nose. This essentially entails the provision of one or more different (gas) sensors which can capture molecules of a smell. On the basis of the signal pattern, a smell 200 (generally learned in advance) can be detected. Since an evaluation unit is generally also integrated in the electronic nose, the smell can be appropriately categorized immediately (without external analysis). Although an electronic nose cannot capture the smell 200 as exactly as the method described above, in which molecules of the smell are bound in a material, a differentiation approximately of the same quality as the human nose is possible. In this way, therefore, desired and undesired smells 200 can be differentiated from one another.

An inquiry may also be made to an occupant if a smell 200 has been detected but it cannot be classified according to whether it is desired or undesired. Thus, this smell 200 may be automatically assigned to the next capture and an appropriate action performed.

In both embodiments (case A manual smell capturing, case B automatic smell capturing), the position of a captured smell 200 may be stored for later use in the form of the position of the vehicle 100 (preferably in a navigation system). Later use may be that a route which includes as many sections as possible having smells 200 categorized as desired is calculated for a specified destination. In the converse case, sections having an undesired smell 200 may also be avoided. It may also be specified for example that the vehicle 100 provides a route that smells as good as possible, for example to go on a weekend trip. This may for example merely involve specifying a duration for the journey and/or a maximum distance in kilometers and/or a destination.

In addition, the time of year may also be used for determining the route, in order for example to take into consideration smells 200 that only occur at certain times of the year.

Furthermore, smells 200 and their position may also be provided for other vehicles 100, either by way of direct V2V communication or by way of corresponding cloud services (V2X). In this way, quicker closing or opening of the ventilation may be performed, that is to say even before the smell 200 is picked up by one's own vehicle 100. It is also possible for a route to be improved, for example by bypassing sections with undesired smells 200 (odors).

Smells 200 may also be assigned to an occupant, in particular a driver. That is to say that if an occupant or a driver has been detected by the vehicle 100 and a smell 200 categorized as desired by them can be emitted by the vehicle 100 (for example because a corresponding smell is present in the fragrance diffuser), this smell 200 is emitted. This may for example take place as a smell of greeting in a welcoming scenario. Actions for smells 200 captured outside the vehicle 1 may also be performed in accordance with the profile of the occupant, since it is known which smells 200 are sensed by the occupant as desired/undesired.

Smells 200 which are categorized according to embodiments of the invention as desired or undesired are smells 200 that can be perceived by the human nose. Smells 200 that cannot be perceived but can harm the human body are not included here. These are already captured by special sensors and an appropriate action is performed.

### DESIGNATIONS

100 Vehicle
200 Smell
300 Smell capturing device, for example smell capturing bell, sensor

1.-10. (canceled)

11. A method for fragrancing an interior of a vehicle based on smells captured from surroundings of the vehicle, the method comprising:
in case A) manually capturing a smell entering an interior of the vehicle by an occupant of the vehicle by way of a smell capturing device, wherein the captured smell is stored and/or analyzed and categorized as desired or undesired by an external facility or directly in the smell capturing device, and/or
in case B) automatically capturing and analyzing a smell occurring outside the vehicle by way of the smell capturing device, wherein the captured smell is categorized as desired or undesired and, based on the categorization, a smell categorized as desired is allowed to enter the interior of the vehicle or a smell categorized as undesired is prevented from entering the interior of the vehicle.

12. The method according to claim 11, wherein in case A) automatic capturing of a smell outside the vehicle is additionally performed, the smell is compared with specified smells and, based on the comparison, the smell is allowed to enter or prevented from entering the interior of the vehicle.

13. The method according to claim 11, wherein a specimen of the stored smell is created for duplication.

14. The method according to claim 13, wherein the specimen is integrated in a fragrance diffuser for use in the vehicle.

15. The method according to claim 11, wherein in case B) the captured and analyzed smell is compared with specified smells and, based on the comparison, the smell is allowed to enter or prevented from entering the interior of the vehicle.

16. The method according to claim 11, wherein an air supply into the interior of the vehicle is opened or increased upon capturing a smell categorized as desired.

17. The method according to claim 11, wherein storing of a position of the captured smell is performed.

18. The method according to claim 17, wherein a route which includes as many sections as possible having smells categorized as desired is calculated for a specified destination.

19. The method according to claim 17, wherein positions of smells categorized as desired or as undesired are provided as information outside the vehicle.

20. A vehicle comprising:
a smell capturing device which is configured to store and/or analyze smells from outside the vehicle, and
a processing facility for carrying out the method according to claim 11.
